(19) United States
(12) Patent Application Publication
(10) Pub. No.: US 2024/0041410 A1
(43) Pub. Date: Feb. 8, 2024

(54) A METHOD AND APPARATUS FOR DETERMINING COMPLIANCE WITH BASELINE BIOMECHANICAL BEHAVIOR

(71) Applicant: Motionize Israel Ltd., Tel Aviv-Yafo (IL)

(72) Inventors: Eran AMIT, Pardes Hanna (IL); Amir Zviran, Tel Aviv (IL); Guy Aharon, Raanana (IL); Moran Gad, Sde Yoav (IL); Erez Morag, Caesarea (IL); Steve Barrett, Hull (GB)

(21) Appl. No.: 18/268,320

(22) PCT Filed: Dec. 20, 2021
(86) PCT No.: PCT/IL2021/051506

§ 371 Date: Jun. 20, 2023

(30) Foreign Application Priority Data
Dec. 20, 2020 (IL) 279613

(51) Int. Cl. A61B 5/00; A61B 5/11; G16H 40/67; G16H 50/20

(52) U.S. Cl. CPC A61B 5/7267; A61B 5/746; A61B 5/112; A61B 5/6807; G16H 40/67; G16H 50/20; A61B 2503/10; A61B 2562/0219

(57) ABSTRACT

A computerized method performed by a processor, an apparatus and a computer program product the method comprising: receiving a model associated with a baseline of one or more biomechanical parameters of one or more action types of a subject, the model describing the biomechanical parameters during a first time period; obtaining one or more values characterizing the biomechanical parameter of the action type in an uncontrolled environment during a second time period, the second time period being later than the first time period; determining whether the value characterizing the biomechanical parameter during the second time period are in compliance with the model; and outputting an alert if the values are not in compliance with the model.

### TECHNICAL FIELD

The present disclosure relates to tracking the behavior of an individual in general, and detecting abnormal biomechanical behavior of the individual, in particular.

### BACKGROUND

Biomechanics is a branch of biophysics that relates to the study of the structure, function and motion of the mechanical aspects of biological systems, at any level from whole organisms to organs, cells and cell organelles, using the methods of mechanics.

Specifically, human biomechanics can be stated as the muscular, joint and skeletal actions of the body during the execution of a given task, skill and/or technique. Human biomechanics may be useful in a plurality of applications, including but not limited to sports and healthcare for people with difficulties or disabilities.

In sports biomechanics, the laws of mechanics are applied to human movement. It focuses on the application of the scientific principles of mechanical physics to understand movements of action of human bodies and sports balls, hockey sticks or the like. Elements of mechanical engineering e.g. strain gauges, electrical engineering e.g. digital filtering, computer science e.g. numerical methods, gait analysis e.g. force platforms, and clinical neurophysiology e.g. surface EMG are some technologies used in human biomechanics.

Proper understanding of biomechanics associated with sports may have implications on sport's performance, injury prevention and rehabilitation, along with sport mastery. For people with disabilities or difficulties, analyzing the human biomechanics can aid in understanding the causes, possibilities, corrective actions or other aspects of healthcare.

### BRIEF SUMMARY

One exemplary embodiment of the disclosed subject matter is a computer-implemented method comprising: receiving a model associated with a baseline of one or more biomechanical parameters of one or more action types of one or more subjects, the model describing a biomechanical parameter during a first time period, wherein the biomechanical parameter is based on a continuous independent motion variable having various values; obtaining one or more values characterizing the biomechanical parameter of the action types in an uncontrolled environment during a second time period, the second time period being later than the first time period; determining whether the values characterizing the biomechanical parameter during the second time period are in compliance with the model; and outputting an alert if the at least one value is not in compliance with the model.

Within the method, obtaining the values characterizing the biomechanical parameter optionally comprises: identifying a plurality of actions from the sensor data; classifying the actions to obtain an action type associated with each action; determining a plurality of points representing the biomechanical parameter for the action type for the subject; and obtaining a characteristic of the plurality of points as the values characterizing the biomechanical parameter. The method can further comprise determining the model, comprising: receiving sensory data of motion by the human subject, the sensory data obtained during the second time period in the uncontrolled environment; identifying a plurality of actions from the sensory data; classifying the plurality of actions to obtain an action type associated with each action; determining one or more pluralities of sensory data measurements representing a biomechanical parameter of the least one action type for the subject; obtaining baseline values characterizing the biomechanical parameters as characterizing values for the pluralities of sensory data measurements; and training the model based on the baseline values characterizing the biomechanical parameters, the model describing the baseline of the action types of the subject. Within the method, the sensory data is optionally obtained from one or more sensors mounted on one or more shoes of the human subject. The method is optionally used for assessing abnormal behavior due to a factor selected from the group consisting of: increase or decrease in physical fitness of the subject; fatigue; injury; a major external variation; and fraud. The method is optionally used for determining that the values characterizing the biomechanical parameter are of a different subject than the subject of the model. The method can further comprise subject to the values being not in compliance with the model: determining that the baseline has changed; and determining a second model to be used instead of the model. Within the method, the values characterizing the biomechanical parameter are optionally described analytically as a function of a continuous independent variable. Within the method, the continuous independent variable is optionally one or more items selected from the group consisting of: linear speed, angular velocity, acceleration, deceleration, jump height or kick velocity. Within the method, the values characterizing the biomechanical parameter optionally comprise a, b and c in a formula of the form: y(x,side)=cₓ e^{-(a+b*side)x} which approximates a collection of (x,y) pairs collected for the subject, wherein side is 1 for one foot and −1 for the other. Within the method, optionally y is a contact duration of a user's foot with the ground, and x is a speed in which the user advances, during straight line running.

Another exemplary embodiment of the disclosed subject matter is an apparatus having a processor, the processor being adapted to perform the steps of: receiving a model associated with a baseline of one or more biomechanical parameters of one or more action types of one or more subjects, the model describing the biomechanical parameters during a first time period, wherein the biomechanical parameter is based on a continuous independent motion variable having various values; obtaining one or more values characterizing the biomechanical parameter of the action types in an uncontrolled environment during a second time period, the second time period being later than the first time period; determining whether the values characterizing the biomechanical parameter during the second time period are in compliance with the model; and outputting an alert if the at least one value is not in compliance with the model.

Within the apparatus, obtaining the values characterizing the biomechanical parameter, optionally comprises: identifying a plurality of actions from the sensor data; classifying the actions to obtain an action type associated with each action; determining a plurality of sensory data measurements representing the biomechanical parameter for the action types for the subject; and obtaining the values characterizing the biomechanical parameter as a characteristic of the plurality of sensory data measurements. Within the apparatus, the processor is optionally further adapted to determine the model, comprising: receiving sensory data of motion by the human subject, the sensory data obtained during the first time period in the uncontrolled environment; identifying a plurality of actions from the sensory data; classifying the actions to obtain an action type associated with each action; determining one or more pluralities of sensory data measurements representing a biomechanical parameter of the action types for the subject; obtaining baseline values characterizing the biomechanical parameter as characterizing the pluralities of sensory data measurements; and training the model based on the baseline values characterizing the biomechanical parameter, the model describing the baseline of the action types of the subjects. Within the apparatus, the sensory data is optionally obtained from one or more sensors mounted on one or more shoes of the human subject. Within the apparatus, the sensory data is optionally obtained from sensors mounted on one or more shoes of the subject and an additional sensor mounted on another location on the human subject. Within the apparatus, the sensory data is optionally obtained from one or more sensors comprising one or more Inertial Measurement Units (IMUs) or a motion capture system. The apparatus is optionally used for assessing abnormal behavior due to a factor selected from the group consisting of: increase or decrease in physical fitness of the subject; fatigue; injury; a major external variation; and fraud. Within the apparatus, subject to the values being not in compliance with the model, the processor is further adapted to: determine that the baseline has changed; and determine a second model to be used instead of the model. Within the apparatus, the values characterizing the biomechanical parameter are optionally described analytically as a function of a continuous independent variable. Within the apparatus, the continuous independent variable is optionally one or more items selected from the group consisting of: linear speed, angular velocity, acceleration, deceleration, jump height or kick velocity. Within the apparatus, the values value characterizing the biomechanical parameter associated with the action types for the subjects, optionally comprise a, b and c in a formula of the form: y(x,side)=cₓ e^{-(a+b*side)x} which approximates a collection of (x,y) pairs collected for the subject, wherein side is 1 for one foot and −1 for the other. Within the apparatus, y is a contact duration of a user's foot with the ground, and x is a speed in which the user advances, during straight line running.

Yet another exemplary embodiment of the disclosed subject matter is a computer program product comprising a computer readable storage medium retaining program instructions, which program instructions when read by a processor, cause the processor to perform a method comprising: receiving a model associated with a baseline of one or more biomechanical parameters of one or more action types of one or more subjects, the model describing the biomechanical parameters during a first time period; obtaining one or more values characterizing the biomechanical parameter of the action types in an uncontrolled environment during a second time period, the second time period being later than the first time period; determining whether the values characterizing the biomechanical parameter during the second time period are in compliance with the model; and outputting an alert if the values are not in compliance with the model.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIGS. 1A and 1B show two views of a footwear sensor unit, in accordance with some exemplary embodiments of the disclosed subject matter;

FIG. 1C shows a shoe having mounted thereon the footwear sensor unit, in accordance with some exemplary embodiments of the disclosed subject matter;

FIGS. 1D-1F show an exemplary housing for a motion sensor, in accordance with some exemplary embodiments of the disclosed subject matter;

FIG. 2A shows a graph demonstrating that different players present similar behavior over a plurality of matches;

FIG. 2B shows a graph demonstrating for each of six players a mean curve, and an area surrounding the mean curve, in accordance with some exemplary embodiments of the disclosed subject matter;

FIGS. 3A and 3B show for a first player and a second player, respectively, a “cloud of points”, each point associated with the contact duration vs. speed at a certain time on one of five different matches, and a representative curve for each match, in accordance with some exemplary embodiments of the disclosed subject matter;

FIG. 4A show for a player a “cloud of points”, each point associated with the flight ratio at a certain time on one of four different matches, and a representative curve for each match, in accordance with some exemplary embodiments of the disclosed subject matter;

FIG. 4B shows a graph displaying approximating curves of flight ratio for six players in four matches, in accordance with some exemplary embodiments of the disclosed subject matter;

FIG. 4C shows for each player a mean curve of flight ratio, and an area containing the curves associated with the player are contained, in accordance with some exemplary embodiments of the disclosed subject matter;

FIG. 4D shows the graph of FIG. 4C after arithmetical adjustments, in accordance with some exemplary embodiments of the disclosed subject matter;

FIGS. 5A, 5B and 5C show graphs of a first, second and third parameters, respectively, representing the biomechanical behavior of five players, in accordance with some exemplary embodiments of the disclosed subject matter;

FIG. 6A shows a flowchart of steps in a method for determining compliance with baseline biomechanical behavior, in accordance with some exemplary embodiments of the disclosed subject matter;

FIG. 6B is a flowchart of steps in an exemplary embodiment of a method for obtaining and using characteristic values of a motion parameter, in accordance with some exemplary embodiments of the disclosed subject matter;

FIG. 7, is a block diagram of a computing platform for determining compliance with baseline biomechanical behavior of a subject, in accordance with some exemplary embodiments of the disclosed subject matter; and

FIG. 8 shows initial experimental results received for a classifier that classifies players in accordance with three parameters, in accordance with some exemplary embodiments of the disclosed subject matter.

### DETAILED DESCRIPTION

In the description below, unless noted otherwise, the term “gait” is to be widely construed as covering a pattern of movement of the limbs of animals, including humans, during locomotion over a solid substrate. The pattern may relate to a single stride or step, or to a plurality thereof.

In the description below, unless noted otherwise, the term “stride” is to be widely construed as covering the movement of one foot during a double step, i.e., from the time the heel of one foot contacts the ground, until the same heel contacts the ground again.

In the description below, unless noted otherwise, the term “step” is to be widely construed as covering a single step, for example from the time one heel forms contact with the ground, until the second heel forms contact with the ground.

In the description below, unless noted otherwise, the terms “subject”, “human subject”, “user”, “player”, “patient”, “healthcare patient” or similar terms are used interchangeably and are all to be widely construed as covering an individual whose biomechanical behavior is examined.

Biomechanics relates to the study of the structure, function and motion of the mechanical aspects of human motion. Of particular interest and need is analysis of the motion performed during sports, or by people with difficulties or disabilities. The study of motion, and in particular human motion may be used to assess and treat individuals with conditions limiting their physical abilities, as well as sportsmen who are always in pursuit of improving their abilities. It will be appreciated that although the discussion below focuses on sportive motions and matches/training sessions, it is equally applicable to healthcare applications and environments.

One technical problem handled by the disclosure relates to assessing and characterizing the performance of a player or a healthcare patient. For example, it may be required to find out whether a player is fit; identify whether he is suffering from an injury; detect short or long term fatigue; differentiate a short term positive or negative spike from long term incremental changes; build and monitor a player profile based on training and matches; give a player real time feedback; validate the identity of a specific player based on his signature for example during scouting, or the like. Similar or different applications may be applied to the healthcare environment, for example assessing the rehabilitation of a person post operation or injury; monitoring the progress of a disease, or the like.

Many factors may influence the behavior of a player during a match or training. Some of these factors depend only on the player's physicality, for example the player's fitness; whether and when he suffered injury; fatigue developing during the match or during the season, or the like. Other factors may be common to the whole or part of a team in the case of a team game, for example whether the match is in the home field or not; which team is dominant; the game strategy, or the like. Some of the team factors may demonstrate greater variability between matches than among the players, in other words one player may behave more like another player in the same match, than like himself in another match.

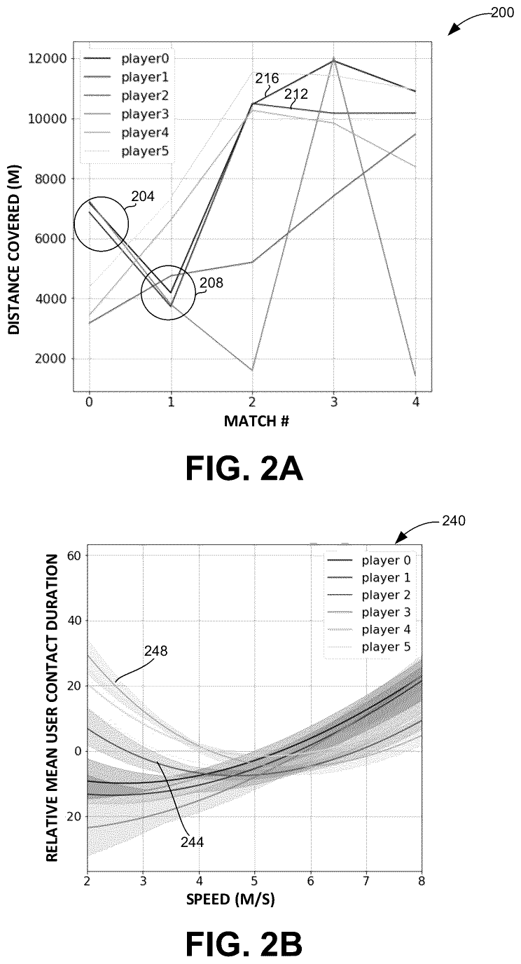

Another technical problem is the limited information that can be gathered from comparing between different players. Referring to FIG. 2A, showing a graph 200 of the distance walked/ran by six players in four matches. It is seen that many players passed distances like each other on one or more events, for example as seen by area 204 related to the first event and area 208 related to the second event.

It is also seen that many of the players' lines show similar behavior for different players, but different behavior for each player in different events. For example, both graphs 212 and 216 start at about 7000 m in the first event, continue with about 4000 m in the second event, about 10500 m in the third event, and 10000-11000 m in the last event. Thus, the variability between the players is less than 10%, while the inter-event variability for the same player is over 100%.

Therefore, it is clear that the external circumstances affect the player's performance to a significant degree, and there may be no point in comparing the player's performance, for example the distance he walks or runs, the number of kicks, or the like between different matches, as this may not be indicative of the player's condition. In addition, comparing the player's performance to the performance of other players may provide little useful information either.

Thus, there is a need to assess a player's physical behavior in the short term and in the long term in a reliable manner that would provide information about the player's physical or mental state, in order to make professional decisions regarding the player, such as devising an appropriate training plan, planning the rehabilitation in case of injury, planning match strategy, or the like. Similarly, in the case of a healthcare patient, it may be required to assess the patient's state, build a plan, give recommendations, or the like.

One technical solution relates to determining how, i.e., in which manner, a human subject performs the actions he takes from a biomechanical point of view, rather than which or how much of these actions he did. The biomechanical behavior is much less prone to vary due to some of the external circumstances (for example, the surface may affect such factors while strategy is less likely to), and can thus provide a stable assessment which may be monitored over time to the performance of the subject. Since the biomechanical behavior may be greatly affected by the human's individual state such as injury, fatigue, mental state, or others, it may thus be an efficient and accurate tool for assessing the human's current state compared to a baseline, and taking decisions based thereon.

Information describing a human motion within a session, such as the behavior of a player in football match, may include many thousands of data points. However, a specific motion can be described by transforming the data points to a few representative characteristics. Some exemplary characteristics include the speed at which a subject's movement changes from walking to running, i.e. the speed at which some of the time both feet are in the air, the speed at which the surface strike changes from toe-strike to heel-strike, the percentage of the time the user's feet are in the air as a function of the running speed, or the like. The characteristics can be represented by a number of parameters, such as a triplet of numbers as disclosed below.

The parameters representing a baseline of the biomechanical behavior may be used for training an artificial intelligence (AI) engine, such as a neural network, a deep neural network, or the like. Subsequently collected parameters may then be provided to the AI engine, which may then output whether the parameters are inline with the training data or indicate a significant change. In some embodiments, the AI engine may output a quantitative measure representing to what degree the parameters comply with the training data. If the parameters are not in compliance with the baseline behavior, a corresponding alert may be provided.

One technical effect of the disclosure relates to creating a baseline for the performance of a human, and comparing later collected data to the baseline, in order to detect significant changes or deviations in how the human is performing. Checking for inconsistency may provide for detecting progress or problems such as injury, fatigue, or the like.

Another technical effect of the disclosure relates to comparing how the human performs the motions relative to past performance, rather than comparing which motions the human performs, thus gaining a more stable indicator to the human's state.

Yet another technical effect of the disclosure relates to comparing the user's biomechanical parameters to the user's baseline biomechanical parameters, rather than or in addition to other individuals such as other team members, because this may not provide a differentiating factor. Even if what the human does is similar to what other users do, for example traverse similar distance during an event, it is how the user does it, which provides a unique signature that may characterize the user and may be monitored over time for detecting changes.

In order to obtain the biomechanical parameter, each human subject may be equipped with one or more sensors mounted on their foot or other body parts, as detailed below.

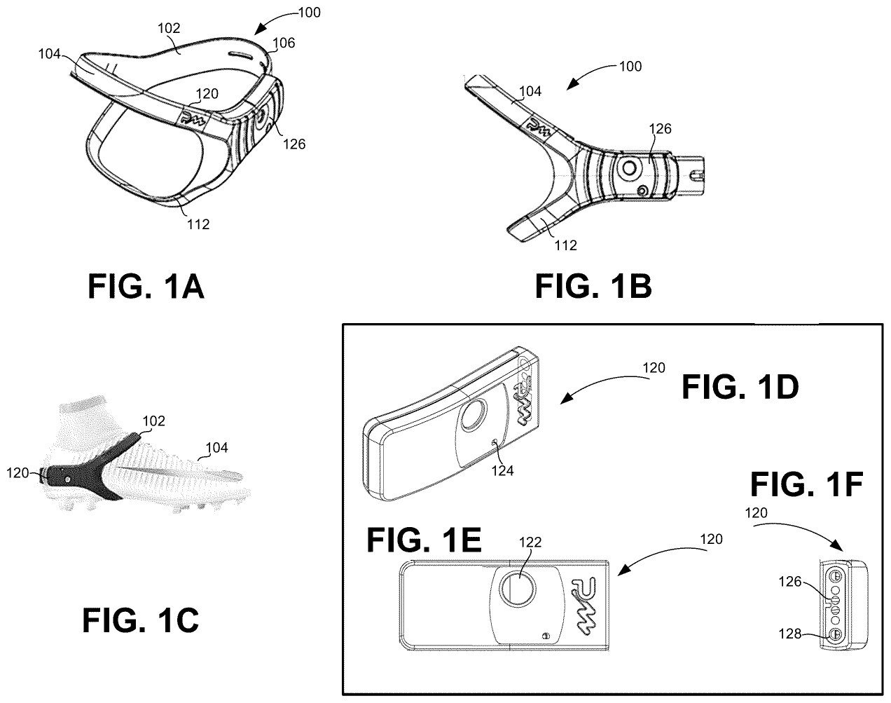

Referring now to FIGS. 1A and 1B showing two views of a footwear sensor unit 100, and to FIG. 1C, showing a shoe 104 having mounted thereon footwear sensor unit 100, and to FIGS. 1D-1F showing an exemplary housing or casing for a motion sensor to be mounted on a shoe.

FIG. 1C depicts a footwear sensor unit 100 mounted on a shoe 104. Footwear sensor unit 100 includes a motion sensor module within a housing 120 and a mounting strap 102. Housing 120 and mounting strap 102 are alternatively referred to herein as a mounting system. Housing 120 may be made of plastic material, while mounting strap 102 may be elastic. Housing 120 may encase a motion sensor, such as a 6 degrees of freedom (DOF) motion sensor, for example an MPU-9150™ made by InvenSense headquartered in San Jose, Calif., USA. The product specification of the MPU-9150™ is incorporated by reference in its entirety and for any purpose. It will be appreciated, however, that the MPU-9150™ is merely an exemplary IMU and that any 6- or 9-DOF or other similar motion sensor can be used. In some embodiments, the motion sensor is a 9-DOF motion sensor of which the system only utilizes sensor data from a 3-axis gyroscope and a 3-axis accelerometer, i.e., only 6-DOF.

Housing 120 may be inserted into a compartment on elastic strap 102. Strap 102 may be mounted on soccer shoe 104.

Strap 102 may be made of any resilient, flexible and elastic material that can be stretched and flexed into place on shoe 104 and withstand the rigors of running, kicking a ball, contact with other players, or the like, while remaining securely in place and not snapping. Strap 102 and housing 120 may be made of rugged, heavy-duty material that is needed to withstand the constant rubbing against the ground (under-strap) and numerous impacts from the soccer ball and other objects such as other players' feet.

Housing 120 may comprise a sensor board, e.g., a printed circuit board (PCB) with components such as the IMU and electronic components. Housing 120 may be designed to keep the PCB safe from any impact it might endure during a match or a training session. Furthermore, the design of strap 102 may place housing 120 in a “ball-free shoe zone”, where the ball is least likely to hit housing 120. Thus, housing 120 may be mounted such that it does not interfere with the way the ball is kicked on one hand, and is not damaged on the other hand. Moreover, strap 102 may be designed in such a manner that all foot movement is directly transferred to the motion sensor as if the foot and the sensor unit formed a single body.

FIGS. 1A and 1B illustrate an exemplary mounting strap 102. Mounting strap 102 may be formed as a single piece of material that includes a back-strap 106, an upper strap 104 and a lower strap 112. Back strap 106 may be U-shaped where the open ends of the U split into upper strap 104 and lower strap 112, both of which make closed circuits of their own. Back strap 106 is adapted to be fitted around the heel of a boot while the front of the boot slips between the upper strap 104 and lower strap 112. Upper strap 104 is adapted to lie across the top rim or near the top rim of the upper of the shoe where the shoe covers the foot, and to cover the part of the shoelaces near the tongue of the shoe. Lower strap 112 may be adapted to be fitted under the bottom of the shoe and to traverse the sole of the boot across an area of the sole which is devoid of cleats (studs). The design, flexibility and elasticity of the mounting strap ensure that the strap is held tightly in place, without shifting position. In embodiments, the strap is adjustable and may or may not form a closed circuit, i.e. two ends of the strap may be open. In other embodiments, the strap is not adjustable. Straps may come in different sizes and can be matched to a given shoe size. The straps can include some or all of the elements described above.

Both right- and left-hand mounting straps may be provided. For a right shoe, the mounting strap may include a holding pouch 126 on the right-hand prong of the U of back strap 106 as viewed from behind the strap and above. For a left foot shoe, holding pouch 126 may be located on the left prong of the U when viewed as above.

FIGS. 1D, 1E and 1F illustrate an exemplary housing 120 for motion sensor 100. Housing 120 may be removably insertable into pouch 126 of strap 102. In the depicted embodiment, housing 120 may include a button aperture 122 via which an operator can actuate the button that activate the motion sensor unit. The casing may further comprise an opening 124 through which one or more LED indicators can be seen. The lights may indicate the status of the motion sensor unit. Other embodiments of the housing may or may not include the same or similar apertures and may or may not have alternative or additional apertures and/or structural elements.

In the depicted embodiment, housing 120 may further include power contacts/ports and/or data ports 128. For example, ports 128 may be power ports for charging a rechargeable battery of the sensor unit and contacts 126 may be data ports for transferring raw or calculated sensor data via a physical medium. Alternatively, ports 128 may be used for data transfer while contacts 126 may be used for charging the unit. In other embodiments, one or more apertures within housing 120 may be used for securing the housing in place while the battery draws charge via contacts 126. The foregoing configurations are merely exemplary and it is made clear that any configuration for charging and/or transferring data is included within the scope of the invention.

Further details of the structure and operation of sensor unit 100 are provided in US Patent Application Publication no. US2020/0229762 published Jul. 23, 2020. In the following description, it is assumed that data has been collected and stored on a server.

Additionally or alternatively, sensory data may be obtained from a motion capture system, such as a camera, a video camera, or the like.

It will be appreciated that the disclosed mounting straps and housing are example only, and that the sensors(s) may be installed anywhere, for example within the shoe sole, on another part of the shoe, as part of another garment such as a sock, or on another part of the user's body, such as the head, hand, torso, lower back, thighs, calves, or the like.

FIG. 2A, as detailed above, demonstrates that different players may take the same actions, meaning that the players' behavior may be significantly influenced by external circumstances related, for example, to the match, rather than by the player himself.

FIG. 2B demonstrates for each player from a group of six players a mean curve, and an area surrounding the mean curve and including the behaviors in a number of events, indicating the mean flight ratio, i.e., the percentage of time the player's leg is above the ground during a stride, vs. the running speed. The description below discloses how this graph can be calculated. The graph shows the curve and the surrounding area for each of the six players, such as curve 244 of player 1 and its surrounding area, and curve 248 of player 4 and its surrounding area. The biomechanical data represented by the graph is collected during one or more matches in which the subject participates, and is used as baseline data for training an AI engine.

When subsequent data becomes available, for example biomechanical data collected during further matches, trainings or other events, the AI engine may provide a yes/no answer or a quantitative indication to whether the player's data is in compliance with the earlier collected data upon which the AI engine was trained. Graphically, this answer indicates whether, or to what degree, a curve describing the later match is within the area associated with the player.

If the answer is negative, or is below a predetermined threshold, it may be appreciated that the subject is in a different state from his baseline state. The state may be better, for example indicating appropriate technical training of the subject, or worse, indicating an injury, fatigue or the like.

In some applications, for example when scouting, a subject may send his data up front. Then, when arriving to the personal assessment, his parameters may be measured and compared to the sent data for authentication and making sure that the subject indeed sent his own data.

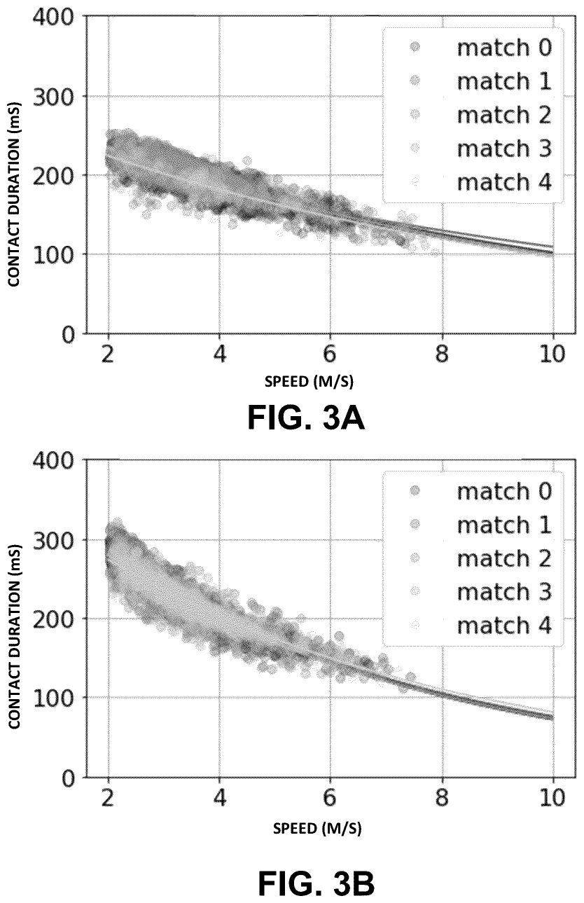

FIG. 3A shows for a first player a “cloud of points”, each point associated with a measurement taken on one of five different matches, each measurement representing the contact duration with the ground vs. the speed, and FIG. 3B demonstrates the same for a second player. It will be appreciated that on each match a plurality of measurements has been taken for each player at different times within the game, thus the cloud of points represents the players' behavior during the match.

FIG. 3A and FIG. 3B also shows a mean curve approximating the points marked for each player for each match. It is seen that the 5 curves which relate to the five matches of player 1 as shown in FIG. 3A are significantly closer to each other, than each of them to the curves of the player 2 for the same match. For instance, the contact duration at speed of 2 m/s is around 230 ms and 280 ms for the first and second players, respectively. Thus, the curves characterize the player, and can be used for assessing the player's identity, state, well being, or the like.

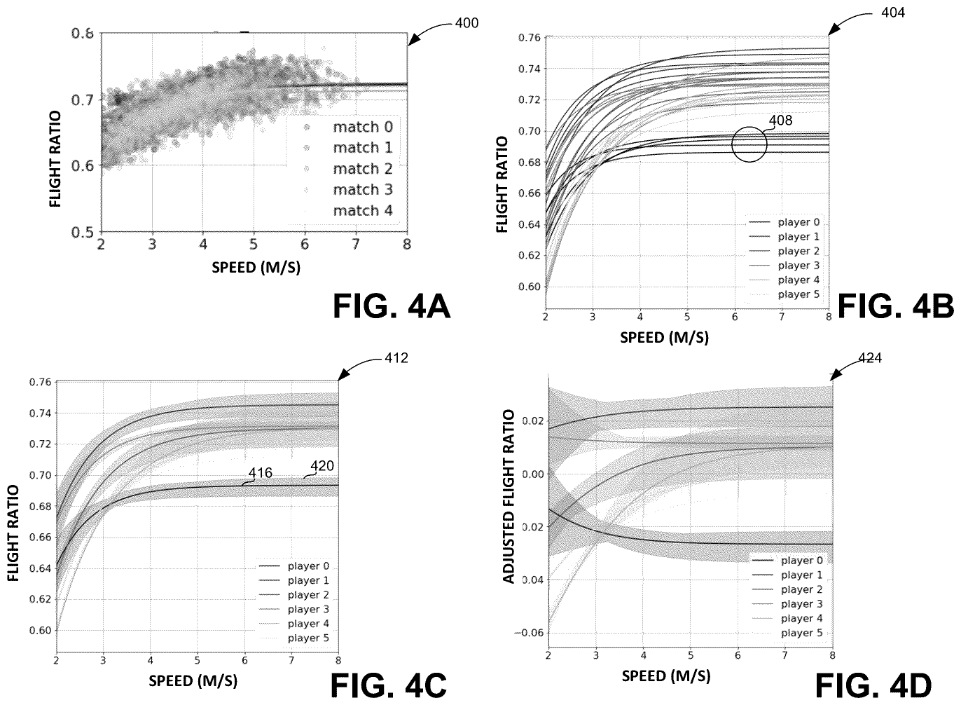

FIG. 4A shows a graph 400 displaying for a first player a plurality of measurements taken on five different matches, each measurement representing the flight ratio vs. the speed, and the approximating curve for each match.

FIG. 4B shows a graph 404 displaying the approximating curves, without the points indicating the measurements, for each of six players for the four matches. It is seen that curves related to the five matches for the same player, for example the five curves related to player 0 pass through area 408.

FIG. 4C shows a graph 412, displaying for each player a mean curve, which is the average of the curves of FIG. 4B associated with the same player, and an area in which all the curves associated with the player are contained. For example, curve 416 is the mean curve for player 0, and grayed area 420 is the corresponding area containing all the curves for player 0.

FIG. 4D shows a graph 424, displaying the same curves and areas as FIG. 3, after arithmetical adjustments, including for example subtracting a typical flight ratio vs. speed curvature from each player curve. In the current example the adjustment is in accordance with the formula 0.72*(1−e^{-1.42*speed}).

Graph 424 thus shows a very clear distinction between the players, such that given another curve for a player, it is easily seen whether the curve is in compliance with the mean curve and area for the player. If the new curve deviates significantly from the area associated with the player, this can indicate a significant positive or negative change in the player's state, a wrong identity of the assumed player, or the like.

Each approximating curve related to each player in each match, as constructed based on the measurements collected for the player, as shown for example on FIGS. 3A, 3B and 4B, may be approximated by an analytical expression. In some exemplary embodiments, the expression may be of the form:

y(x,side)=c*e^{-(a+b*side)x} or of the form y(x,side)=c*(1−e^{-(a+b*side)x})

which approximate a collection of the (x,y) pairs of which the curve constitutes, wherein side is 1 for one foot and −1 for the other. Thus, given the curve approximating the collection of points indicating the parameters of the player in a subsequent event, a, b and c may be calculated for each player for each match, training or during everyday life. The (a, b, c) triplets for each player and each match may be used for training an AI engine, such as a neural network. When a triplet is obtained for a further match or training, the AI engine may provide an answer or a probability of whether a given triplet is inline with the training data. It will be appreciated that each such triplet is representative of a specific biomechanical characteristic in a specific action type. The triplet represents the subject motion mechanism while performing the action, and may include asymmetric behavior, as indicated by parameter b.

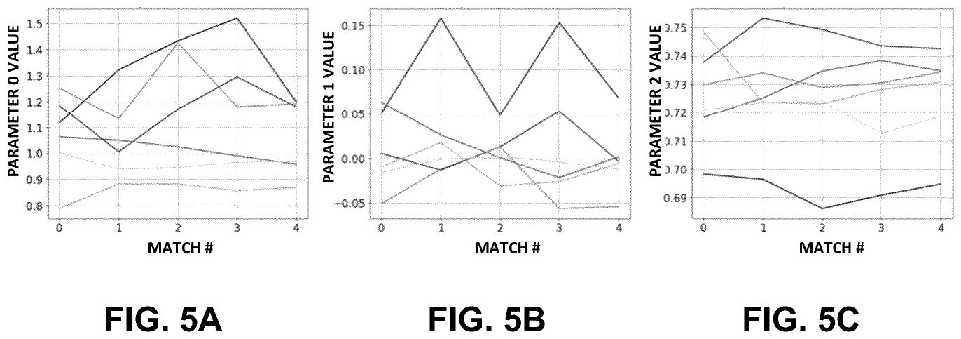

FIG. 5A shows the value of a for each of five players for five matches, and similarly FIGS. 5B and 5C show the b and c values. It will also be appreciated that an AI engine may be trained upon and adapted to receive a plurality of characteristic sets representative of a plurality of action types of a player, representing a biomechanical signature.

The triplet values, the biomechanical characteristic, the action type and others may be obtained for each player and each match using, for example, regression methods or other algorithms.

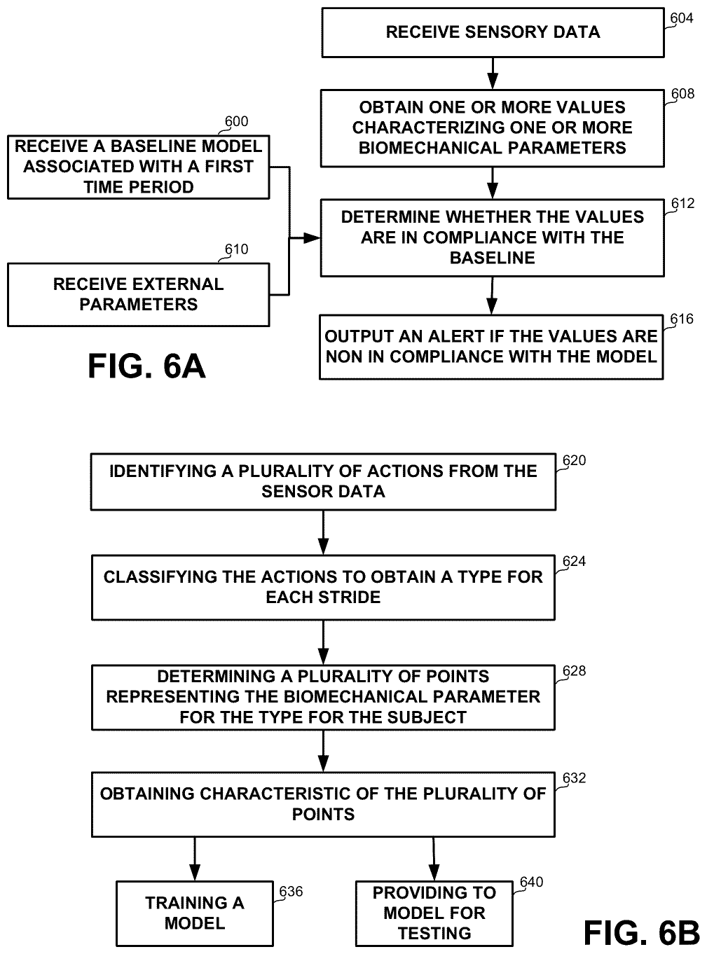

Referring now to FIG. 6A, showing a flowchart of steps in a method for determining compliance with baseline biomechanical behavior, in accordance with the disclosure.

On step 600, a model of the motion of one or more subjects may be received, the model associated with a baseline of one or more biomechanical parameters of one or more action types of the subject(s), the baseline describing the biomechanical parameter as captured during a first time period. In some embodiments, the model may be based upon data obtained in an uncontrolled environment, such as a sports match, a sports training session, a motion by a healthcare patient, or the like. However, the data or some of it may also be obtained in a controlled environment such as a laboratory, a physician's office, or the like. The model may be an AI engine, such as a neural network, a deep neural network, or the like. In some embodiments, where the model comprises information related to a plurality of subjects, the model may be useful in separating global effects from personal effects.

FIG. 6B and the associated disclosure demonstrate an embodiment of constructing the obtained model.

Once trained, the model, or a module using the model may be configured to receive one or more values, and determine whether the values are in compliance with the model, i.e. with the baseline of the biomechanical parameters. The compliance result may be binary, e.g., complying/non-complying, or numeric, in which case it may be determined whether the result is below or above a compliance threshold. In some embodiments, the training data may comprise sets of parameters which when substituted in a formula describe a characteristic behavior of the biomechanical parameter during the first time period.

On step 604, sensory data may be obtained, for example from one or more sensor units mounted on one or more subjects, for example the sensor units described in association with FIGS. 1A-1F above. The sensory data may be collected during a second time period, which may be later than the time period. The sensory data may be obtained in an uncontrolled environment.

On step 608, one or more values characterizing the biomechanical parameter during the second time period may be obtained from the sensory data. In some embodiments, the values may be parameters which when substituted in a formula describe a characteristic behavior of the biomechanical parameter.

On step 610, specific parameters that may influence the model's decision may be received, such as weather conditions, surface condition, or the like.

On step 612, it may be determined whether the one or more values comply with the model, e.g., a binary or a numeric value may be received as described above. It will be appreciated that the model represents a baseline of the characteristic biomechanical behavior, based on one or more action types. Thus, incompliance may be detected by the deviation of a combination of the action type parameters from the model, rather than the deviation of one or more individual parameters.

On step 616, if the one or more values are non-compliant with the model, for example the binary result is negative (or positive, depending on how the result is interpreted), or a numeric compliance value is below a threshold, then an alert may be output. Such situation may occur, for example, due to fatigue, injury, or the like. In further situations, the non-compliance may be due to fraud, for example a player providing measurements or a model of another subject, and provides lesser performance when tested personally.

In some situations, for example if multiple sets of values of a subject are non-s compliant with the model, it may be deduced that the baseline, e.g. the performance of the subject has changed. In such case, a more updated model may be determined for the subject, based at least on the new data or on a combination of old and new data.

In some embodiments, a plurality of indications related to the subject may be provided, which relate to different biomechanical parameters. The different compliance results or scores for different parameters may help a user, such as a coach, a physician, or others assess the subject's state, progress, health, or the like.

Referring now to FIG. 6B, showing a flowchart of steps in an exemplary embodiment of a method for obtaining and using characteristic values of a motion parameter.

On step 620, a plurality of actions such as strides, ball touches or others may be obtained from the sensor data received on step 604. On step 624 the actions may be classified according to action type, to identify actions associated with one or more actions types of interest, such as straight run, left turn while running, right turn while running, straight walk, jump, kick, or the like.

Exemplary embodiments of steps 620 and 624 are provided in U.S. patent application Ser. No. 17/063,132 titled “Method and Apparatus for gait Analysis” filed Oct. 5, 2020 and assigned to the same assignee as the current application.

On step 628, a plurality of points in one, two or more dimensions, representing biomechanical parameters for the subject and for the actions type may be obtained from the sensor data. The biomechanical parameters may be, for example, different kinematic properties as a function of speed: contact duration, flight ration, flight time, max foot height, stride and step length, stride and step duration, pronation and respective leg asymmetries, or the like. Additional parameters may include maximal body height and body angle.

Some characteristics may be obtained directly from the sensor raw signal, for example: maximal angular velocity at one or more axes as a function of speed or kick velocity, angular velocity during toe off, maximal acceleration in different axes vs. speed, or the like.

Other independent parameters may be angular velocity, acceleration, deceleration and jump height, wherein each parameter(s) may be used in a different action type.

Although the discussion above is more focused on motion types, it will be appreciated that it is equally applicable to sport specific behavior such as ball touch technique. For example, the action types may relate to the ball touch type, such as kick, pass, receive, or local.

Further kinematic properties may be a function of kick speed, such as: maximal foot height, contact time or contact angle of second foot, used foot zone, second foot rotation, difference between the legs, or the like.

Additional parameters may relate to the gait mechanism, such as but not limited to the speed at which a player modifies his surface strike mechanism from heel-strike to toe-strike, or changes from walking to running.

On step 632, one or more characteristics of the plurality of points may be obtained, e.g., the “a,b,c” triplets per each characteristic as described in association with FIGS. 4D, 5A, 5B and 5C above. It will be appreciated, however, that one or more action type characteristics may be described analytically as a function of one or more continuous independent variables, such as but not limited to linear speed, angular velocity, acceleration, deceleration, jump height, kick velocity, or the like.

It will be appreciated that one or more sets of characteristics may be obtained, for example a set per each match per player.

On step 636 the plurality of sets may be used as baseline, for training an AI engine. In some embodiments, an AI engine may be trained per parameter, per player, per action type, while in other embodiments a model may relate to a plurality of parameters, a plurality of players, or a plurality of action types.

The engine maybe further adapted to receive and to consider specific parameters as part of its decisions, such as weather conditions, surface, or the like.

On step 640, one set of characteristics may be provided to an AI engine trained for the corresponding player and action type, and the AI engine may indicate whether or to what degree the set of characteristic is in compliance with the model.

To improve the model performance in detecting specific variations, baseline parameters of multiples sessions can be recorded. For example—at the beginning and at the and of a training session for quantifying fatigue effects.

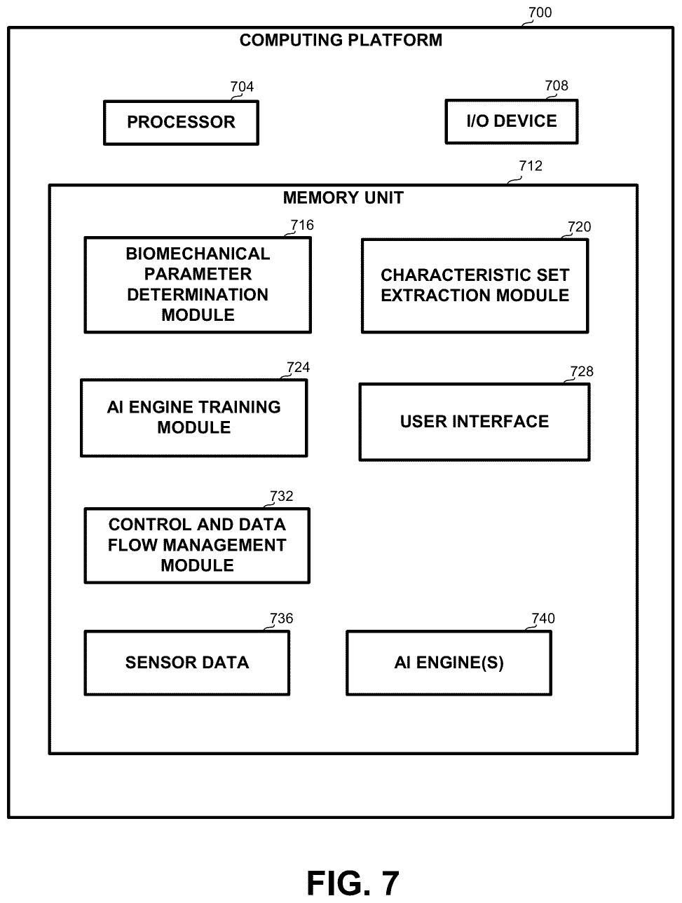

Referring now to FIG. 7, showing a block diagram of a computing platform 700 for determining compliance with baseline biomechanical behavior of a subject, in accordance with some exemplary embodiments of the disclosed subject matter.

It will be appreciated that one or more of the components detailed below may be executed by a remote processor communicating with computing platform 700, such as a remote server. In the discussion below the term “user” may relate to an individual examining the performance of a subject, such as the subject himself, a coach, a physician or the like. The remote server may receive information and provide results to multiple instances of computing platforms 700, whether associated with the same user or subject, with different users, different subjects acting together such as a sports team, or from unrelated users or subjects. In some embodiments, a hybrid approach may be taken, in which initial calculations are performed in real time by a computing platform embedded within the device. The calculations may then be improved by a remote computing platform, upon data received form additional sensors and/or subjects.

In some exemplary embodiments computing platform 700 may comprise a processor 704, which may be a Central Processing Unit (CPU), a microprocessor, an electronic circuit, an Integrated Circuit (IC) or the like. Processor 704 may be utilized to perform computations required by computing platform 700 or any of its subcomponents. Processor 704 may be configured to execute computer-programs useful in performing the methods of FIGS. 6A and 6B above.

In some exemplary embodiments, one or more I/O devices 708 may be configured to receive input from and provide output to a user. In some exemplary embodiments, I/O devices 708 may be utilized to present to the user a user interface, obtain user definitions, and display the results such as weather the subject behavior as expressed by the biomechanical parameters is inline with the model. I/O devices 708 may comprise a display, a keyboard, a mouse, a touch screen or another pointing or tracking device, a voice activated module, or the like.

In some exemplary embodiments computing platform 700 may comprise a memory unit 712. Memory unit 712 may be a short-term storage device or long-term storage device. Memory unit 712 may be a persistent storage or volatile storage. Memory unit 712 may be a disk drive, a Flash disk, a Random Access Memory (RAM), a memory chip, or the like. Memory unit 712 may be a single memory device, or multiple interconnected memory devices which may be co-located or located in different locations and communicating via any communication channel. Memory unit 712 may retain one or more databases

In some exemplary embodiments, memory unit 712 may retain program code operative to cause processor 704 to perform acts associated with any of the subcomponents of computing platform 700. In some exemplary embodiments, memory unit 512 may retain program code operative to cause processor 704 to perform acts associated with any of the steps shown in FIG. 6 above.

The components detailed below may be implemented as one or more sets of interrelated computer instructions, executed for example by processor 704 or by another processor. The components may be arranged as one or more executable files, dynamic libraries, static libraries, methods, functions, services, or the like, programmed in any programming language and under any computing environment.

Memory unit 712 may retain biomechanical parameter determination module 716, for receiving sensor data and outputting a collection of values or value sets characterizing biomechanical parameters associated with one or more action types for one or more users.

Biomechanical parameter determination module 716 may use components detailed in U.S. patent application Ser. No. 17/063,132 titled “Method and Apparatus for gait Analysis” filed Oct. 5, 2020 and assigned to the same assignee as the current application, for identifying the data related to the required action type, and may also comprise modules for calculating the specific values or value sets representing the biomechanical parameters.

Memory unit 712 may retain characteristic set extraction module 720, for obtaining one or more values characterizing the biomechanical parameters determine by biomechanical parameter determination module 716. For example, characteristic set extraction module 720 may calculate parameters defining a curve approximating the biomechanical parameters determined by biomechanical parameter determination module 716. The operation of biomechanical parameter determination module 716 and characteristic set extraction module 720 is described in association with steps 608, 620, 624 and 628 above.

Memory unit 712 may retain AI engine training module 724 for receiving one or more characteristic sets and training an AI engine upon the characteristic sets, as described in association with step 636 above.

Memory unit 712 may retain user interface 728 for receiving instructions or preferences from a user, and providing information to the user over I/O device 708.

Memory unit 712 may retain data and control flow management module 732 for activating and managing the control flow between the various modules, obtaining and providing the data required for each module, or the like.

Memory unit 712 may retain sensor data 736 comprising readings obtained for example from the sensor units of FIGS. 1A-1F, or processing products thereof such as characteristic values of a motion parameter for a plurality of action types, plurality of players, plurality of events, or the like.

Memory unit 712 may retain one or more AI engines 740, trained upon one or more characteristic sets, and configured to receive another characteristic set and output a determination whether the characteristic set complies with the training data.

Experimental Results

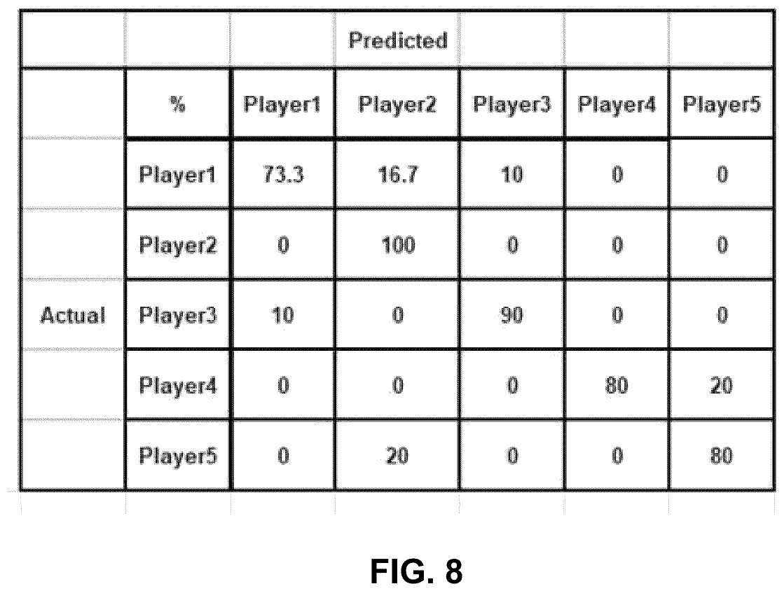

FIG. 8 shows initial experimental results received for a classifier that classifies players according to three biomechanical parameters: typical contact time at speed of 2 and 8 m/s and flight time decay vs. speed, which is parameter a in the triplets discussed above. The model was trained and tested using five matches and 10 permutations, such that every run used a different combination of two matches for training and three matches for testing. The training accuracy, i.e. the results of running the model on the training set, was 94% with a standard deviation of 7%, and the test accuracy was 85% with a standard deviation of 7%, which is very high considering the small exemplary training set. A correct result, i.e. the numbers along the main diagonal, indicates no significant change in the biomechanical parameters of the player. A wrong answer, such as the confusion between player 5 and player 2 may indicate insufficient training, similarity between the players or insufficient model parameters, since only 3 parameters were used. Since in a real working environment the identity of a player is known, a “correct identification” may indicate no significant deviation in the subject's performance relative to the training set, and vice versa.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as Python, MATLAB, the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computerized method performed by a processor, comprising:
receiving a model associated with a baseline of at least one biomechanical parameter of at least one action type of at least one subject, the model describing the at least one biomechanical parameter during a first time period, wherein the biomechanical parameter is based on a continuous independent motion variable having various values;
obtaining at least one value characterizing the biomechanical parameter of the at least one action type in an uncontrolled environment during a second time period, the second time period being later than the first time period;
determining whether the at least one value characterizing the biomechanical parameter during the second time period are in compliance with the model; and
outputting an alert if the at least one value is not in compliance with the model.

2. The method of claim 1, wherein obtaining the at least one value characterizing the biomechanical parameter, comprises:
identifying a plurality of actions from the sensor data;
classifying the actions to obtain an action type associated with each action;
determining a plurality of points representing the biomechanical parameter for the at least one action type for the subject; and
obtaining a characteristic of the plurality of points as the at least one value characterizing the biomechanical parameter.

3. The method of claim 1, further comprising determining the model, comprising:
receiving sensory data of motion by the human subject, the sensory data obtained during the second time period in the uncontrolled environment;
identifying a plurality of actions from the sensory data;
classifying the plurality of actions to obtain an action type associated with each action;
determining at least one plurality of sensory data measurements representing a biomechanical parameter of the least one action type for the subject;
obtaining at least one baseline value characterizing the biomechanical parameter as characterizing values for the at least one plurality of sensory data measurements; and
training the model based on the at least one baseline value, the model describing the baseline of the at least one action type of the subject.

4. The method of claim 1, wherein the sensory data is obtained from at least one sensor mounted on at least one shoe of the human subject.

5. The method of claim 1, wherein the method is used for assessing abnormal behavior due to a factor selected from the group consisting of: increase or decrease in physical fitness of the subject; fatigue; injury; a major external variation; and fraud.

6. The method of claim 1, wherein the method is used for determining that the at least one value characterizing the biomechanical parameter are of a different subject than the subject of the model.

7. The method of claim 1, further comprising subject to the at least one value being not in compliance with the model:
determining that the baseline has changed; and
determining a second model to be used instead of the model.

8. The method of claim 1, wherein the at least one value characterizing the biomechanical parameter is described analytically as a function of a continuous independent variable.

9. The method of claim 8, wherein the continuous independent variable is at least one item selected from the group consisting of: linear speed, angular velocity, acceleration, deceleration, jump height or kick velocity.

10. The method of claim 1, wherein the at least one value characterizing the biomechanical parameter is associated with the at least one action type for the subject comprise a, b and c in a formula of the form:
y(x,side)=cₓe^{-(a*b*side)x}
which approximates a collection of (x,y) pairs collected for the subject, wherein side is 1 for one foot and −1 for the other.

11. The method of claim 10, wherein
y is a contact duration of a user's foot with the ground, and
x is a speed in which the user advances, during straight line running.

12. An apparatus having a processor, the processor being adapted to perform the steps of:
receiving a model associated with a baseline of at least one biomechanical parameter of at least one action type of at least one subject, the model describing the at least one biomechanical parameter during a first time period wherein the biomechanical parameter is based on a continuous independent motion variable having various values;
obtaining at least one value characterizing the biomechanical parameter of the at least one action type in an uncontrolled environment during a second time period, the second time period being later than the first time period;
determining whether the at least one value characterizing the biomechanical parameter during the second time period are in compliance with the model; and
outputting an alert if the at least one value is not in compliance with the model.

13. The apparatus of claim 12, wherein obtaining the at least one value characterizing the biomechanical parameter, comprises:
identifying a plurality of actions from the sensor data;
classifying the actions to obtain an action type associated with each action;
determining a plurality of sensory data measurements representing the biomechanical parameter for the at least one action type for the subject; and
obtaining the at least one value characterizing the biomechanical parameter as a characteristic of the plurality of sensory data measurements.

14. The apparatus of claim 12, wherein the processor is further adapted to determine the model, comprising:
receiving sensory data of motion by the human subject, the sensory data obtained during the first time period in the uncontrolled environment;
identifying a plurality of actions from the sensory data;
classifying the actions to obtain an action type associated with each action;
determining at least one plurality of sensory data measurements representing a biomechanical parameter of the least one action type for the subject;
obtaining the at least one baseline value characterizing the biomechanical parameter as characterizing the at least one plurality of sensory data measurements; and
training the model based on the at least one baseline value characterizing the biomechanical parameter, the model describing the baseline of the at least one action type of the subject.

15. The apparatus of claim 12, wherein the sensory data is obtained from at least one sensor mounted on at least one shoe of the human subject.

16. The apparatus of claim 12, wherein the sensory data is obtained from sensors mounted on at least one shoe of the subject and an additional sensor mounted on another location on the human subject.

17. The apparatus of claim 12, wherein the sensory data is obtained from at least one sensor comprising at least one Inertial Measurement Unit (IMU) or a motion capture system.

18. The apparatus of claim 12, wherein the apparatus is used for assessing abnormal behavior due to a factor selected from the group consisting of: increase or decrease in physical fitness of the subject; fatigue; injury; a major external variation; and fraud.

19. The apparatus of claim 12, wherein subject to the at least one value being not in compliance with the model, the processor is further adapted to:
determine that the baseline has changed; and
determine a second model to be used instead of the model.

20. The apparatus of claim 12, wherein the at least one value characterizing the biomechanical parameter is described analytically as a function of a continuous independent variable.

21. The apparatus of claim 20, wherein the continuous independent variable is at least one item selected from the group consisting of: linear speed, angular velocity, acceleration, deceleration, jump height or kick velocity.

22. The apparatus of claim 12, wherein the at least one value characterizing the biomechanical parameter associated with the at least one action type for the subject comprises a, b and c in a formula of the form:
y(x,side)=cₓe^{-(a+b*side)x}
which approximates a collection of (x,y) pairs collected for the subject, wherein side is 1 for one foot and −1 for the other,

23. The apparatus of claim 17, wherein
y is a contact duration of a user's foot with the ground, and
x is a speed in which the user advances, during straight line running.

24. A computer program product comprising a computer readable storage medium retaining program instructions, which program instructions when read by a processor, cause the processor to perform a method comprising:
receiving sensory data of motion by the human subject, the sensory data obtained during a first time period in an uncontrolled environment with a continuous independent motion variable having various values;
identifying a plurality of actions from the sensory data;
classifying the actions to obtain an action type associated with each action;
determining at least one plurality of sensory data measurements representing a biomechanical parameter of the least one action type for the subject;
obtaining the at least one value of the biomechanical parameter as characterizing the at least one plurality of sensory data measurements; and
training the model based on the at least one value of the biomechanical parameter, the model describing the baseline of the at least one action type of the subject.
